(19) United States
(12) Patent Application Publication
(10) Pub. No.: US 2024/0099959 A1
(43) Pub. Date: Mar. 28, 2024

(54) OIL-IN-WATER-TYPE LIP COSMETIC

(71) Applicant: Shiseido Company, Ltd., Tokyo, (JP)

(72) Inventors: Mika KONDO, Tokyo (JP); Kyohei KOSAKA, Tokyo (JP); Akiko FUKUI, Tokyo (JP); Ayumi ASAI, Tokyo (JP); Kiriko CHIBA, Tokyo (JP); Ayano KIGAWA, Tokyo (JP)

(73) Assignee: Shiseido Company, Ltd., Tokyo, (JP)

(21) Appl. No.: 18/275,935

(22) PCT Filed: Sep. 13, 2021
(86) PCT No.: PCT/JP2021/033559

§ 371 Date: Aug. 4, 2023

(30) Foreign Application Priority Data
Mar. 5, 2021 (JP) 2021-034999

(51) Int. Cl. A61K 8/891; A61K 8/81; A61Q 1/06

(52) U.S. Cl. CPC A61K 8/891; A61K 8/8117; A61Q 1/06; A61K 2800/43; A61K 2800/48

(57) ABSTRACT

The present invention is to provide an oil-in-water type lip cosmetic having an excellent secondary adhesion-less effect and excellent in gloss feeling, and fit feeling for the lips and fresh.

Oil-in-water type lip cosmetic excellent not only in secondary adhesion-less effect but also in gloss feeling and fit feeling is obtained by combining a dimethyl silicone oil having a particular viscosity with a film-forming agent including a hydrocarbon-based polymer incompatible with the silicone oil.

### RELATED APPLICATION

The present application claims priority to Japanese Patent Application No. 2021-34999 filed Mar. 5, 2021, the disclosure of which is hereby incorporated by reference.

### FIELD OF THE INVENTION

The present invention relates to a lip cosmetic and particularly to a lip cosmetic having an excellent secondary adhesion-less effect and excellent in gloss persistence and fit feeling.

### BACKGROUND OF THE INVENTION

Conventionally, for lip cosmetics, secondary adhesiveness in which the cosmetic is transferred when the lips come into contact with a cup or the like after the cosmetic is applied has been a problem, and lip cosmetics less likely to cause secondary adhesion and having the so-called secondary adhesion-less effect have also been developed.

For example, Patent Literature 1 discloses a solid cosmetic having transfer resistance in which a silicone-based surfactant is blended in combination with a volatile oil, and a pigment is well dispersed.

Patent Literature 2 describes a lipstick composition containing a perfluoropolyether type nonvolatile oil and a volatile oil that are incompatible, and having transfer resistance. But a problem of this lipstick composition is that it takes some time after application to exhibit a secondary adhesion-less effect.

### CITATION LIST

### Patent Literature

[Patent Literature 1] International Publication No. 97/16157
[Patent Literature 2] International Publication No. 96/40044

### SUMMARY OF INVENTION

### Problem to be Solved by the Invention

The present invention has been made in view of the conventional art, and a problem thereof to be solved is to provide an oil-in-water type lip cosmetic having an excellent secondary adhesion-less effect and excellent in gloss feeling, and fit feeling for the lips and fresh.

### Means to Solve the Problem

The present inventors have studied diligently in order to solve the problem, and as a result found that an oil-in-water type lip cosmetic excellent not only in secondary adhesion-less effect but also in gloss feeling and fit feeling is obtained by combining a dimethyl silicone oil having a particular viscosity with a film-forming agent including a hydrocarbon-based polymer incompatible with the silicone oil. Thus, the present inventors have completed the present invention.

An oil-in-water type lip cosmetic, comprising the following (A) to (E) components:
(A) a dimethyl silicone oil having a viscosity of 100 CS to 50000 CS
(B) a film-forming agent comprising a hydrocarbon-based polymer incompatible with the (A)
(C) a non-silicone-based surfactant having an HLB of 12 or more
(D) a water-based thickening agent
(E) water.

An oil-in-water type lip cosmetic in the present invention is preferable that the hydrocarbon-based polymer of the film-forming agent is 0.5 to 10% by mass in the cosmetic.

It is preferable in the present invention that the film-forming agent comprises a hydrocarbon-based solvent for dissolving the hydrocarbon-based polymer.

It is preferable in the present invention that the hydrocarbon-based solvent is 50 to 99 parts by weight based on 100 parts by weight of the film-forming agent.

It is preferable in the present invention that the amounts of the (A) to (E) components blended are
(A) dimethyl silicone oil having a viscosity of 100 CS to 50000 CS 10 to 40% by mass
(B) film-forming agent comprising the hydrocarbon-based polymer incompatible with (A) 5 to 30% by mass
(C) non-silicone-based surfactant having an HLB of 12 or more 1 to 4% by mass (D) water-based thickening agent 0.01 to 5% by mass
(E) water 25 to 70% by mass.

It is preferable in the present invention that the hydrocarbon-based polymer is selected from an ethylene monomer unit and/or a styrene monomer unit.

It is preferable in the present invention that the (D) water-based thickening agent is a water-based thickening agent comprising an acrylic acid-based polymer.

It is preferable in the present invention that no pigment is comprised.

It is preferable in the present invention to comprise 0.01 to 1.5% by mass of (F) an aqueous dye.

It is preferable in the present invention to comprise (G) a moisturizer.

It is preferable in the present invention that the (G) moisturizer is blended 0.1 to 5% by mass of an alkylene oxide derivative represented by the following formula (1) which has a molecular weight of 1000 to 5000,

[Formula 1]

R¹O—[(AO)ₚ(EO)_{q}]—R² (1)

wherein AO is an oxyalkylene group having 3 to 4 carbon atoms, EO is an oxyethylene group, p and q are respectively average addition mole numbers of the oxyalkylene group having 3 to 4 carbon atoms and the oxyethylene group, 1≤p≤70, 1≤q≤70, a ratio of the oxyethylene group to a total of the oxyalkylene group and the oxyethylene group is in a range of 20 to 80% by mass,
the oxyalkylene group and the oxyethylene group may be added in a block form or in a random form, R¹ and R² are hydrocarbon groups having 1 to 4 carbon atoms that may be the same or different, or hydrogen atoms, and a ratio of a number of hydrogen atoms to a number of hydrocarbon groups for R¹ and R² is 0.15 or less.

### Effect of the Invention

For the oil-in-water type lip cosmetic according to the present invention, by blending particular amounts of the (A) dimethyl silicone oil having a viscosity of 100 CS to 50000 CS, the (B) film-forming agent including the hydrocarbon-based polymer incompatible with the (A) component, the (C) non-silicone-based surfactant having an HLB of 12 or more, the (D) water-based thickening agent, and the (E) water, an oil-in-water type lip cosmetic that exhibits a secondary adhesion-less effect from immediately after application and is excellent in gloss and fit feeling is obtained.

### DESCRIPTION OF THE PREFERRED EMBODIMENT

A lip cosmetic according to the present invention includes (A) a dimethyl silicone oil having a viscosity of 100 CS to 50000 CS, (B) a film-forming agent including a hydrocarbon-based polymer incompatible with the (A) component, (C) a non-silicone-based surfactant having an HLB of 12 or more, (D) a water-based thickening agent, and (E) water.

The components will be described in detail below.

(A) Dimethyl Silicone Oil Having Viscosity of 100 CS to 50000 CS

The (A) component is an oil incompatible with the (B) component. The (A) component has a low affinity for the lips, and therefore when the lip cosmetic is applied to the lips, the (A) component forms the outermost phase, and the (A) component is attached to a cup or the like, and thus the color is not attached to the cup, and the so-called “secondary adhesion-less effect” is exhibited. The viscosity of the (A) component is preferably 100 CS to 50000 CS. Further, the viscosity is particularly preferably 500 CS to 30000 CS. Even a mixture of dimethyl silicone oils having different viscosities can be used when the viscosities are within this range. When the viscosity is 100 CS or less, the secondary adhesion-less effect may not be exhibited. When the viscosity is more than 50000 CS, a thick impression may be made.

Specific examples of the (A) component include dimethicone 100 CS, dimethicone 5000 CS, dimethicone 500 CS, dimethicone 1000 CS, and dimethicone 3000 CS. Commercial products include KF-96A-300cs, KF-96A-500cs, and KF-96A-5000cs (all are manufactured by Shin-Etsu Chemical Co., Ltd.).

The amount of the (A) component blended is preferably 10 to 40% by mass based on the entire cosmetic. When the amount blended is less than 10% by mass, separation is insufficient at the time of application, and the gloss may be insufficient. When the amount blended is more than 40% by mass, the fresh feeling of use is impaired.

(B) Film-Forming Agent Including Hydrocarbon-Based Polymer Incompatible with (A) Component

The (B) component is a film-forming agent including a hydrocarbon-based polymer incompatible with the (A) component. This (B) component has a higher affinity for the lips than the (A) component and easily closely adheres to the lips. Therefore, the (B) component forms a film between the lips and the (A) component, and thus a fit feeling is felt, and the secondary adhesion-less effect can be more expected than when there is no film.

Examples of the hydrocarbon-based polymer blended into the lip cosmetic of the present invention include one or two or more selected from copolymers having at least one selected from an ethylene monomer unit and a styrene monomer unit. For example, the hydrocarbon-based polymer can include at least one selected from a (styrene/isoprene) copolymer, an (ethylene/propylene/styrene) copolymer, a (styrene/butadiene) copolymer, a (butylene/ethylene/styrene) copolymer, a (styrene/propylene/butylene) copolymer, a (styrene/butylene) copolymer, and an (ethylene/propylene) copolymer. Among them, particularly an (ethylene/propylene) copolymer, an (ethylene/propylene/styrene) copolymer, and a (butylene/ethylene/styrene) copolymer are preferred.

In the present invention, the film-forming agent preferably includes a hydrocarbon-based solvent as a solvent for dissolving the hydrocarbon-based polymer. When no hydrocarbon-based solvent is used, the spreadability on the skin may be poor with only the hydrocarbon-based polymer. As the hydrocarbon-based solvent, isododecane, hydrogenated polyisobutene, and the like are preferred.

For the hydrocarbon-based solvent, either a volatile solvent or a nonvolatile solvent can be used.

When a volatile solvent is used, the hydrocarbon-based solvent volatilizes over time after application, and the hydrocarbon-based polymer forms a firm film.

When a nonvolatile solvent is used, a moist gel-like film is formed on an application surface by the hydrocarbon-based polymer and the hydrocarbon-based solvent.

For the concentration of the hydrocarbon-based solvent in the film-forming agent, the hydrocarbon-based solvent is preferably 50 to 99 parts by mass based on 100 parts by mass of the film-forming agent.

Examples of commercial products that can be used as the film-forming agent include VERSAGEL® MD1600 and ME2000 (Penreco) and Creagel® Crystal ID and Crystal AF.

The amount of the hydrocarbon-based polymer blended in the cosmetic is preferably 0.5 to 10% by mass, particularly preferably 1 to 7 parts by mass. When the amount of the hydrocarbon-based polymer blended is less than 0.5% by mass, a film-forming effect on an application surface may not be sufficiently obtained. When the amount of the hydrocarbon-based polymer blended is more than 10% by mass, the feeling of use, such as stickiness, may be influenced.

The amount blended in the cosmetic as the film-forming agent is preferably 5 to 30% by mass. When the amount blended is less than 5% by mass, a film may not be formed. When the amount blended is more than 30% by mass, the fresh feeling of use may be impaired.

(C) Non-Silicone-Based Surfactant Having HLB of 12 or More

The lip cosmetic includes a non-silicone-based surfactant having an HLB of 12 or more as the (C) component. The HLB of the surfactant is 12 or more, and thus the surfactant dissolves in water, and an oil-in-water type composition is obtained. Further, a non-silicone-based activator is used because when (F) an aqueous dye mixes with the (A) silicone oil, the secondary adhesion-less effect is not obtained.

Examples of the non-silicone-based surfactant having an HLB of 12 or more include ceteth-20, PEG-60 hydrogenated castor oil, beheneth-20, and PEG-20 glyceryl isostearate.

Commercial products include NIKKOL BC-20, NIKKOL HCO-60, and NIKKOL BB-20 (the above are manufactured by Nikko Chemicals Co., Ltd.) and EMALEX GWIS-120 (manufactured by Nihon Emulsion Co., Ltd.).

One or two or more (C) components may be blended into the lip cosmetic of the present invention.

The amount of the (C) component blended is preferably 1 to 4% by mass based on the entire cosmetic. When the amount blended is less than 1% by mass, the effect of the (C) component may not be exhibited. When the amount blended is 4% by mass or more, the secondary adhesion-less effect may decrease.

(D) Water-Based Thickening Agent

In this application, a water-based thickening agent can be included as the (D) component. The lip cosmetic preferably includes the (D) component in terms of stable emulsification. Examples of the (D) component include (dimethylacrylamide/acryloyldimethyltaurine Na) crosspolymers, (acrylates/alkyl acrylate (C10-30)) crosspolymers, and (hydroxyethyl acrylate/acryloyldimethyltaurine Na) copolymers. Among them, particularly water-based thickening agents including acrylic acid-based polymers are preferred.

Examples of commercial products include SIMULGEL™ NS (manufactured by SEPPIC S.A.).

When the (D) component is blended, the amount blended is preferably 0.01 to 5% by mass based on the total amount of the lip cosmetic. When the amount blended is less than 0.01% by mass, the (D) component may not contribute to the stability of emulsification. When the amount blended is more than 5% by mass, the fresh feeling of use may be impaired.

(E) Water

The oil-in-water type lip cosmetic according to the present invention includes water. The water is not particularly limited and may be ion-exchanged water, tap water, or the like.

The amount of the (E) water blended is preferably 25 to 70% by mass based on the total amount of the oil-in-water type lip cosmetic. When the amount blended is less than 25% by mass, the smoothness at the time of use may be impaired. When the amount blended is more than 70% by mass, the amounts of other components blended decrease relatively, and the effects are not obtained.

(F) Aqueous Dye

The oil-in-water type lip cosmetic according to the present invention can further include the (F) aqueous dye. With the oil-in-water type lip cosmetic according to the present invention, the secondary adhesion-less effect is obtained by the fact that the coloring matter produces a color by infiltrating the lips, rather than in a state of being in the cosmetic applied to the lips, and therefore an aqueous dye is used.

Examples of the (F) component include acid fuchsin, Yellow No. 4, Yellow No. 5, Blue No. 1, Red No. 227, Green No. 3, Red No. 401, Red No. 504, Orange No. 205, Yellow No. 203, Green No. 201, Violet No. 201, Violet No. 401, Black No. 401, and Yellow No. 205.

Commercial products include UNICERT Yellow 08006-J (manufactured by Sensient Technologies Japan Co., Ltd.) and FD&C Green No. 3 (manufactured by Sensient Technologies Japan Co., Ltd.).

When the (F) component is blended, the amount of the (F) component blended is preferably 0.01 to 1.5% by mass based on the total amount of the cosmetic. When the amount of the (F) component blended is less than 0.01% by mass, the effect of the aqueous dye is not obtained. When the amount of the (F) component blended is more than 1.5% by mass, the cosmetic may be difficult to remove.

(G) Moisturizer

The oil-in-water type lip cosmetic according to the present invention can further include (G) a moisturizer. With the oil-in-water type lip cosmetic according to the present invention, a dry feeling may be felt due to the volatilization of water, and this can be suppressed by blending a moisturizer.

Examples of the (G) moisturizer include moisturizers usually blended into cosmetics, for example, alkylene oxide derivatives, ethylene glycol, diethylene glycol, 1,3-butylene glycol, glycerin, hexamethylene glycol, isoprene glycol, polyethylene glycol, hyaluronic acid, chondroitin sulfuric acid, chitin, chitosan, xylitol, sorbitol, maltitol, mucoitin sulfuric acid, caronic acid, atelocollagen, cholesteryl-12-hydroxystearate, sodium lactate, bile salts, dl-pyrrolidone carboxylate, short chain soluble collagen, diglycerin (EO) PO adducts, chestnut rose extracts, yarrow extracts, and melilot extracts.

The (G) moisturizer preferably has an alkylene oxide derivative having a molecular weight of 1000 to 5000.

The alkylene oxide derivative is not particularly limited but is preferably a compound represented by the following formula (1):

[Formula 1]

R¹O—[(AO)ₚ(EO)_{q}]—R² (1)

wherein AO is an oxyalkylene group having 3 to 4 carbon atoms, EO is an oxyethylene group, p and q are respectively average addition mole numbers of the oxyalkylene group having 3 to 4 carbon atoms and the oxyethylene group, 1≤p≤70, 1≤q≤70, a ratio of the oxyethylene group to a total of the oxyalkylene group and the oxyethylene group is in a range of 20 to 80% by mass,
the oxyalkylene group and the oxyethylene group may be added in a block form or in a random form, R¹ and R² are hydrocarbon groups having 1 to 4 carbon atoms that may be the same or different, or hydrogen atoms, and a ratio of a number of hydrogen atoms to a number of hydrocarbon groups for R¹ and R² is 0.15 or less.

In the compound represented by the formula (1), AO is an oxyalkylene group having 3 to 4 carbon atoms, and examples thereof include an oxypropylene group, an oxybutylene group, an oxyisobutylene group, a trimethylene group, and a tetramethylene group, preferably an oxypropylene group or an oxybutylene group. p is the average addition mole number of the oxyalkylene group having 3 to 4 carbon atoms, 1≤p≤70, and preferably 2≤p≤20. q is the average addition mole number of the oxyethylene group, 1≤q≤70, and preferably 2 q 20. (p+q) is preferably 8 to 100.

In the compound represented by the formula (1), the ratio of the oxyethylene group to the total of the oxyalkylene group having 3 to 4 carbon atoms and the oxyethylene group is preferably in the range of 20 to 80% by mass. The order of addition of ethylene oxide and an alkylene oxide having 3 to 4 carbon atoms is not particularly limited. The oxyethylene group and the oxyalkylene group having 3 to 4 carbon atoms may be added in a block form or in a random form but are preferably added in a random form. Here, the block form includes not only a double block but also a triple block.

For R¹ and R², the same type may be used, a hydrocarbon group having 1 to 4 carbon atoms and a hydrogen atom may coexist, or different types of hydrocarbon groups having 1 to 4 carbon atoms may coexist, provided that for the abundance ratio between the hydrocarbon group and the hydrogen atom, of the hydrocarbon groups of R¹ and R², the ratio of the number of hydrogen atoms (Y) to the number of hydrocarbon groups (X), Y/X, is 0.15 or less, preferably 0.06 or less.

Specific examples of the alkylene oxide derivative include polyoxyethylene (10 mol) polyoxypropylene (10 mol) dimethyl ether, polyoxyethylene (9 mol) polyoxypropylene (2 mol) dimethyl ether, polyoxyethylene (14 mol) polyoxypropylene (7 mol) dimethyl ether, polyoxyethylene (6 mol) polyoxypropylene (14 mol) dimethyl ether, polyoxyethylene (15 mol) polyoxypropylene (5 mol) dimethyl ether, polyoxyethylene (25 mol) polyoxypropylene (25 mol) dimethyl ether, polyoxyethylene (9 mol) polyoxybutylene (2 mol) dimethyl ether, polyoxyethylene (14 mol) polyoxybutylene (7 mol) dimethyl ether, polyoxyethylene (10 mol) polyoxypropylene (10 mol) diethyl ether, polyoxyethylene (10 mol) polyoxypropylene (10 mol) dipropyl ether, polyoxyethylene (10 mol) polyoxypropylene (10 mol) dibutyl ether, and polyoxyethylene (36 mol) polyoxypropylene (41 mol) dimethyl ether. Among these, polyoxyethylene (9 mol) polyoxypropylene (2 mol) dimethyl ether, polyoxyethylene (14 mol) polyoxypropylene (7 mol) dimethyl ether, or polyoxyethylene (36 mol) polyoxypropylene (41 mol) dimethyl ether is preferred, and polyoxyethylene (9 mol) polyoxypropylene (2 mol) dimethyl ether is particularly preferred.

The alkylene oxide derivative of the present invention can be produced by a known method. For example, the alkylene oxide derivative of the present invention can be obtained by addition-polymerizing ethylene oxide and an alkylene oxide having 3 to 4 carbon atoms to a compound having a hydroxyl group, and then subjecting an alkyl halide to an ether reaction in the presence of an alkali catalyst.

The amount blended of the alkylene oxide derivative included in the cosmetic of the present invention is not particularly limited but is preferably 0.1 to 5% by mass based on the total amount of the cosmetic, more preferably 0.5 to 2% by mass based on the total amount of the cosmetic.

In addition to the components, an oil agent, a powder, a polymer compound, a perfume, an antioxidant, a preservative, a beauty component, and the like other than the above, used in ordinary oil-in-water type lip cosmetics can be appropriately blended into the oil-in-water type lip cosmetic of the present invention in a range that does not impair the effect of the present invention.

The oil-in-water-type lip cosmetic of the present invention may be applied to a lip gloss, a lip primer, an overcoat for lipsticks, and a lip cream. In particular, a lip gloss is preferred.

The present invention will be described with reference to Examples below, but the present invention is not limited by those Examples. The amount blended is in % by mass unless otherwise specified.

The evaluations in the Examples were performed as follows.

### (1) Appearance

### (Method of Evaluating Appearance)

For appearance uniformity after sample making, five-grade evaluation was performed based on the following scoring criteria. From the score average value, a determination was made according to the following evaluation criteria.

### (Score)

3: uniform
2: there is a somewhat nonuniform part
1: nonuniform

### (Evaluation Criteria)

A: Average score of 2.0 or more
B: Average score of 1.5 or more and less than 2.0
C: Average score of less than 1.5

### (2) Secondary Adhesion Resistance

### (Method of Evaluating Secondary Adhesion Resistance)

A practical usability test was performed by a panel of 10 experts. The secondary adhesion resistance effect when the sample was applied to lips, five-grade evaluation was performed based on the following scoring criteria. From the average score, determination was made based on the following evaluation criteria.

### (Score)

3: a high secondary adhesion-less effect is felt
2: secondary adhesion effectless is felt
1: no secondary adhesion effectless is felt

### (Evaluation Criteria)

A: Average score of 2.0 or more
B: Average score of 1.5 or more and less than 2.0
C: Average score of less than 1.5

### (3) Gloss

### (Method of Evaluating Gloss)

A practical usability test was performed by a panel of 10 experts. Gloss when the sample was applied to lips, three-grade evaluation was performed based on the following scoring criteria. From the average score, determination was made based on the following evaluation criteria.

### (Score)

3: a moderate gloss is felt
2: the gloss is not enough
1: no gloss is felt
   (Evaluation criteria)
A: Average score of 2.0 or more
B: Average score of 1.5 or more and less than 2.0
C: Average score of less than 1.5

### (4) Fit Feeling

### (Method of Evaluating Fit Feeling)

An actual usability test by a 10-member expert panel was performed. For a fit feeling when a sample was applied to the lips, three-grade evaluation was performed based on the following scoring criteria. From the score average value, a determination was made according to the following evaluation criteria.

### (Score)

3: a moderate fit feeling is felt
2: the fit feeling is not enough
1: no fit feeling is felt

### (Evaluation Criteria)

A: Average score of 2.0 or more
B: Average score of 1.5 or more and less than 2.0
C: Average score of less than 1.5

### (5) Moist Feeling

### (Evaluation of Moist Feeling)

An actual usability test by a 10-member expert panel was performed. For the moist feeling of the lips 4 h after a sample was applied to the lips, three-grade evaluation was performed based on the following scoring criteria. From the score average value, a determination was made according to the following evaluation criteria.

### (Score)

3: a moist feeling is felt
2: the moist feeling is not enough
1: no moist feeling is felt

### (Evaluation Criteria)

A: Average score of 2.0 or more
B: Average score of 1.5 or more and less than 2.0
C: Average score of less than 1.5

First, the inventors studied whether (A) a dimethyl silicone oil was blended or not and further the viscosity of the (A) dimethyl silicone oil. The results are shown in Table 1.

TABLE 1

| | Test Example | 1-1 | 1-2 | 1-3 |
|---|---|---|---|---|
| Silicone Oil | Dimethicone 5000 CS | 28 | | |
| | Dimethicone100 CS | | | 28 |
| Film-forming | (Ethylene/propylene) copolymer (35 parts by | 20 | 20 | 20 |
| agent | weight) Isododecane (65 parts by weight) | | | |
| | Amount of polymer | 7 | 7 | 7 |
| Surfacant | Ceteth-20 | 0.5 | 0.5 | 0.5 |
| | PEG-60 hydrogenated castor oil | 2 | 2 | 2 |
| Water-besed | (Dimethylacrylamide/acloyldimethyltaurine | 0.05 | 0.05 | 0.05 |
| thicking agent | Na) crosspolymer | | | |
| | (Acrylates/alkyl acrylate (C10-30)) | 0.027 | 0.027 | 0.027 |
| | crosspolymer | | | |
| | (Hydroxyethyl | 0.9 | 0.9 | 0.9 |
| | acrylate/acryloyldimethyltaurine Na) | | | |
| | copolymer | | | |
| Water | Ion-exchanged water | 35.338 | 63.338 | 35.338 |
| Aqueous dye | Acis fuchsin | 0.025 | 0.025 | 0.025 |
| | Yellow No. 4 | 0.027 | 0.027 | 0.027 |
| Moisturizer | 1,3-Butylene glycol | 7 | 7 | 7 |
| Others | K cocoyl glutamete | 0.1 | 0.1 | 0.1 |
| | Ethanol | 5.2 | 5.2 | 5.2 |
| | Caustic potash | 0.035 | 0.035 | 0.035 |
| | Bentonite | 0.798 | 0.798 | 0.798 |
| Evaluation | Appearance | A | A | A |
| | Secondary adhesion | A | B | A |
| | Gloss | A | C | B |
| | Fit feeling | A | B | B |
| | Moist feeling over time | A | B | A |

As seen from Table 1, whether the dimethyl silicone oil was 5000 CS or 100 CS, an oil-in-water type lip cosmetic having both a good appearance and good usability was obtained (test examples 1-1 and 1-3). But when no dimethyl silicone oil was blended as in test example 1-2, the gloss feeling was insufficient.

Next, the inventors studied the amount of the dimethyl silicone oil blended. The results are shown in Table 2.

TABLE 2

| | Test example | 1-1 | 2-1 | 2-2 |
|---|---|---|---|---|
| Silicone oil | Dimethicone5000 CS | 28 | 10 | 5 |
| Film-forming | (Ethylene/propylene) copolymer (35 parts by | 20 | 20 | 20 |
| agent | weight) Isododecane (65 parts by weight) | | | |
| | Amount of polymer | 7 | 7 | 7 |
| Surfactant | Ceteth-20 | 0.5 | 0.5 | 0.5 |
| | PEG-60 hydrogenated castor oil | 2 | 2 | 2 |
| Water-based | (Dimethylacrylamide/acloyldimethyltaurine | 0.05 | 0.05 | 0.05 |
| thickening agent | Na) crosspolymer | | | |
| | (Acrylates/alkyl acrylate (C10-30)) | 0.027 | 0.027 | 0.027 |
| | crosspolymer | | | |
| | (Hydroxyethyl | 0.9 | 0.9 | 0.9 |
| | acrylate/acryloyldimethyltaurine Na) | | | |
| | copolymer | | | |
| Water | Ion-exchanged water | 35.338 | 53.338 | 58.338 |
| Aqueous dye | Acid fuchsin | 0.025 | 0.025 | 0.025 |
| | Yellow No. 4 | 0.027 | 0.027 | 0.027 |
| Moisturizer | 1,3-Butylene glycol | 7 | 7 | 7 |
| Others | K cocoyl glutamate | 0.1 | 0.1 | 0.1 |
| | Ethanol | 5.2 | 5.2 | 5.2 |
| | Caustic potash | 0.035 | 0.035 | 0.035 |
| | Bentonite | 0.798 | 0.798 | 0.798 |
| Evaluation | Appearance | A | A | A |
| | Secondary adhesion | A | A | A |
| | Gloss | A | B | C |
| | Fit feeling | A | A | A |
| | Moist feelinf over time | A | A | A |

As seen from Table 2, when 28% by mass of the dimethyl silicone oil was included, a cosmetic good in all items was obtained (test example 1-1). When the amount of the dimethyl silicone oil blended was 5% by mass, no gloss feeling was produced (test example 2-2). As a result of a further detailed study, it was found that a preferred amount of the dimethyl silicone oil blended was 10 to 40% by mass.

Next, the inventors studied the type and amount of (B) a film-forming agent including a hydrocarbon-based polymer incompatible with (A). The results are shown in Table 3.

TABLE 3

| | Test Example | 1-1 | 3-1 | 3-2 | 3-3 | 3-4 | 3-5 |
|---|---|---|---|---|---|---|---|
| Silicone oil | Dimethicone5000 CS | 28 | 28 | 28 | 28 | 28 | 28 |
| Film-forming | (Ethylene/propylene) copolymer (35 parts by | 20 | 10 | 5 | | | |
| agent | weight) Isododecane (65 parts by weight) | | | | | | |
| | (Ethylene/propylene/styrene) copolymer + | | | | 20 | | |
| | (butylene/ethylene/styrene) copolymer (2.5 | | | | | | |
| | parts by mass) Isododecane (97.5 parts by | | | | | | |
| | mass) | | | | | | |
| | (Ethylene/propylene/styrene) copolymer + | | | | | 10 | |
| | (butylene/ethylene/styrene) copolymer (35 | | | | | | |
| | parts by mass) Hydrogenated | | | | | | |
| | polyisobutene (65 parts by mass) | | | | | | |
| | Trimethylsiloxysilicic acid (50 parts by mass) | | | | | | 10 |
| | Isododecane (50 parts by mass) | | | | | | |
| | Amount of polymer | 7 | 3.5 | 1.75 | 0.5 | 3.5 | 5 |
| Surfactant | Ceteth-20 | 0.5 | 0.5 | 0.5 | 0.5 | 0.5 | 0.5 |
| | PEG-60 hydrogenated castor oil | 2 | 2 | 2 | 2 | 2 | 2 |
| Water-based | (Dimethylacrylamide/acloyldimethyltaurine | 0.05 | 0.05 | 0.05 | 0.05 | 0.05 | 0.05 |
| Thickening | Na) crosspolymer | | | | | | |
| agent | (Acrylates/alkyl acrylate (C10-30)) | 0.027 | 0.027 | 0.027 | 0.027 | 0.027 | 0.027 |
| | crosspolymer | | | | | | |
| | (Hydroxyethyl | 0.9 | 0.9 | 0.9 | 0.9 | 0.9 | 0.9 |
| | acrylate/acryloyldimethyltaurine Na) | | | | | | |
| | copolymer | | | | | | |
| Water | Ion-exchanged water | 35.338 | 45.338 | 50.338 | 35.338 | 45.338 | 45.338 |
| Aqueous dye | Acid fuchsin | 0.025 | 0.025 | 0.025 | 0.025 | 0.025 | 0.025 |
| | Yellow No. 4 | 0.027 | 0.027 | 0.027 | 0.027 | 0.027 | 0.027 |
| Moisturizer | 1,3-Butylene glycol | 7 | 7 | 7 | 7 | 7 | 7 |
| Others | K cocoyl glutamate | 0.1 | 0.1 | 0.1 | 0.1 | 0.1 | 0.1 |
| | Ethanol | 5.2 | 5.2 | 5.2 | 5.2 | 5.2 | 5.2 |
| | Caustic potash | 0.035 | 0.035 | 0.035 | 0.035 | 0.035 | 0.035 |
| | Bentonite | 0.798 | 0.798 | 0.798 | 0.798 | 0.798 | 0.798 |
| Evaluation | Appearance | A | A | A | A | A | A |
| | Secondary adhesion | A | A | A | A | A | C |
| | Gloss | A | A | A | A | A | B |
| | Fit feeling | A | A | A | B | A | B |
| | Moist feeling over time | A | A | A | A | A | C |

As seen from Table 3, with a film-forming agent including a hydrocarbon-based polymer, an oil-in-water type lip cosmetic having both a good appearance and a good feeling of use was obtained even if the type of the film-forming agent was different (test examples 1-1, 3-3, and 3-4). However, even with an oily film-forming agent, a sufficient secondary adhesiveness-improving effect was not noted with trimethylsiloxysilicic acid, and it was suggested that the effect was specific to hydrocarbon-based polymers.

Isododecane used as the hydrocarbon-based solvent of a film-forming agent is volatile, and hydrogenated polyisobutene is nonvolatile. In the comparison of test examples 3-1 and 3-4, an excellent cosmetic was obtained when either solvent was used. When volatile isododecane was used, a firm film was formed. When nonvolatile hydrogenated polyisobutene was used, a flexible, moist gel-like film was formed.

When test examples 3-2 and 3-3 are compared, it is understood that particularly when the amount of a hydrocarbon-based polymer is 0.5% by mass or more in a cosmetic, an excellent feeling of use is obtained.

As a result of a further detailed study, it became clear that the amount of a hydrocarbon-based polymer blended in a cosmetic was preferably 0.5 to 10% by mass, particularly preferably 1 to 7% by mass.

The amount of a film-forming agent including a hydrocarbon-based polymer and a hydrocarbon-based solvent blended in a cosmetic is preferably 5 to 30% by mass.

A hydrocarbon-based solvent in a film-forming agent is preferably 50 to 99 parts by mass from the viewpoint of producibility, and spreadability at the time of application.

Further, the inventors compared an oil-in-water type lip cosmetic obtained when (F) an aqueous dye was added, and an oil-in-water type lip cosmetic obtained when a color material was added. The results are shown in Table 4.

TABLE 4

| | | | |
|---|---|---|---|
| Silicone oil | Dimethicone5000 CS | 28 | 28 |
| Film-forming | (Ethylene/propylene) copolymer (35 parts by | 20 | 20 |
| agent | weight) Isododecane (65 parts by weight) | | |
| | Amount of polymer | 7 | 7 |
| Surfactant | Ceteth-20 | 0.5 | 0.5 |
| | PEG-60 hydrogenated castor oil | 2 | 2 |
| Water-based | (Dimethylacrylamide/acloyldimethyltaurine | 0.05 | 0.05 |
| thickening agent | Na) crosspolymer | | |
| | (Acrylates/alkyl acrylate (C10-30)) | 0.027 | 0.027 |
| | crosspolymer | | |
| | (Hydroxyethyl | 0.9 | 0.9 |
| | acrylate/acryloyldimethyltaurine Na) | | |
| | copolymer | | |
| Water | Ion-exchanged water | 35.338 | 35.29 |
| Aqueous dye | Acid fuchsin | 0.025 | |
| | Yellow No. 4 | 0.027 | |
| Oil dispersible | Red No. 202 | | 0.1 |
| pigment | | | |
| Moisturizer | 1,3-Butylene glycol | 7 | 7 |
| Others | K cocoyl glutamate | 0.1 | 0.1 |
| | Ethanol | 5.2 | 5.2 |
| | Caustic potash | 0.035 | 0.035 |
| | Bentonite | 0.798 | 0.798 |
| Evaluation | Appearance | A | C |
| | Secondary adhesion | A | C |
| | Gloss | A | A |
| | Fit feeling | A | A |
| | Moist feeling over time | A | A |

As is clear from Table 4, when an oil-dispersible color material was added to an oil-in-water type lip cosmetic according to the present invention, the secondary adhesion-less effect disappeared, and the appearance was also nonuniform (test example 4-1).

### Formulation Example

Formulation Example 1: Liquid type lip stick

| | Compounding |
|---|---|
| Component | Amount (% by mass) |
| Ion exchanged water | Amount |
| 1,3-Butylene glycol | 7.0 |
| Ceteth-20 | 0.5 |
| PEG-60 hydrogenated castor oil | 2.0 |
| (Ethylene/propylene) copolymer + Isododecane | 20.0 |
| Hydrogenated polyisobutene | 3.0 |
| Dimethicone (5000 CS) | 28.0 |
| Ethanol | 5.0 |
| Acid fuchsin | 0.03 |
| Yellow No. 4 | 0.03 |
| (Dimethylacrylamide/acloyldimethyltaurine | 0.05 |
| Na) crosspolymer | |
| (Acrylates/alkyl acrylate (C10-30)) crosspolymer | 0.1 |
| (Hydroxyethyl acrylate/acryloyldimethyltaurine | 0.9 |
| Na) copolymer | |
| Potasium hydroxide | 0.04 |
| Bentonite | 0.8 |
| PEG/PPG-36/41 Dimethylethel | 1.0 |
| Chestnut rose fruit extract | 0.1 |
| Niacinamid | 0.1 |

1. An oil-in-water type lip cosmetic, comprising the following (A) to (E) components:
(A) a dimethyl silicone oil having a viscosity of 100 CS to 50000 CS
(B) a film-forming agent comprising a hydrocarbon-based polymer incompatible with the (A)
(C) a non-silicone-based surfactant having an HLB of 12 or more
(D) a water-based thickening agent
(E) water.

2. The cosmetic according to claim 1, wherein
the hydrocarbon-based polymer of the film-forming agent is 0.5 to 10% by mass in the cosmetic.

3. The cosmetic according to claim 2, wherein the film-forming agent comprises a hydrocarbon-based solvent for dissolving the hydrocarbon-based polymer.

4. The cosmetic according to claim 3, wherein the hydrocarbon-based solvent is 50 to 99 parts by weight based on 100 parts by weight of the film-forming agent.

5. The oil-in-water type lip cosmetic according to claim 1, wherein amounts of the (A) to (E) components blended are
(A) dimethyl silicone oil having a viscosity of 100 CS to 50000 CS 10 to 40% by mass
(B) film-forming agent comprising the hydrocarbon-based polymer incompatible with (A) and the hydrocarbon-based solvent 5 to 30% by mass
(C) non-silicone-based surfactant having an HLB of 12 or more 1 to 4% by mass
(D) water-based thickening agent 0.01 to 5% by mass
(E) water 25 to 70% by mass.

6. The oil-in-water type lip cosmetic according to claim 1, wherein (B) hydrocarbon-based polymer is selected from an ethylene monomer unit and/or a styrene monomer unit.

7. The oil-in-water type lip cosmetic according to claim 1, wherein (D) water-based thickening agent is a water-based thickening agent comprising an acrylic acid-based polymer.

9. The oil-in-water type lip cosmetic according to claim 1, comprising no pigment.

9. The oil-in-water type lip cosmetic according to claim 1, comprising 0.01 to 1.5% by mass of (F) aqueous dye.

10. The oil-in-water type lip cosmetic according to claim 1, further comprising (G) moisturizer.

11. The oil-in-water type lip cosmetic according to claim 10, wherein (G) moisturizer is blended 0.1 to 5% by mass of an alkylene oxide derivative represented by the following formula (1) which has a molecular weight of 1000 to 5000,
[Formula 1]
R¹O—[(AO)ₚ(EO)_{q}]—R² (1)
wherein AO is an oxyalkylene group having 3 to 4 carbon atoms, EO is an oxyethylene group, p and q are respectively average addition mole numbers of the oxyalkylene group having 3 to 4 carbon atoms and the oxyethylene group, 1≤p≤70, 1≤q≤70, a ratio of the oxyethylene group to a total of the oxyalkylene group and the oxyethylene group is in a range of 20 to 80% by mass,
the oxyalkylene group and the oxyethylene group may be added in a block form or in a random form, R¹ and R² are hydrocarbon groups having 1 to 4 carbon atoms that may be the same or different, or hydrogen atoms, and a ratio of a number of hydrogen atoms to a number of hydrocarbon groups for R¹ and R² is 0.15 or less.
